(19) United States
(12) Patent Application Publication
(10) Pub. No.: US 2020/0073110 A1
(43) Pub. Date: Mar. 5, 2020

(54) SURGICAL LOUPE

(71) Applicant: SONY CORPORATION, TOKYO (JP)

(72) Inventors: TAKESHI MAEDA, TOKYO (JP); YOHEI KURODA, TOKYO (JP)

(21) Appl. No.: 16/466,374

(22) PCT Filed: Oct. 10, 2017
(86) PCT No.: PCT/JP2017/036582

§ 371 Date: Jun. 4, 2019

(30) Foreign Application Priority Data
Dec. 26, 2016 (JP) 2016-251113

(51) Int. Cl. G02B 25/00; G02C 7/08; G02B 7/06; G02B 25/02; A61B 90/25; A61B 90/35

(52) U.S. Cl. CPC G02B 25/004; G02C 7/088; G02B 7/06; A61B 2090/061; A61B 90/25; A61B 90/35; G02B 25/02

(57) ABSTRACT

[Object] To provide a surgical loupe that can adjust a convergence angle more easily.

[Solution] A surgical loupe including: two optical systems that cause images of light from a surgical field which is an observation target to be formed on eyes of a wearer; and a drive unit for adjusting a convergence angle formed by optical axes of the two optical systems.

### TECHNICAL FIELD

The present disclosure relates to a surgical loupe.

### BACKGROUND ART

Delicate treatments have been demanded in surgical operations, for example, cardiovascular surgery, in recent years. When such surgery is performed, operators need to observe objects in stereoscopic and enlarged views, and thus they often use surgical loupes which are binocular magnifying glasses.

Patent Literature 1, for example, discloses a surgical loupe that provides an enlarged image of an observation target to the eyes of a wearer who is a user and wirelessly transmits a captured image obtained through imaging by an imager to the outside.

### CITATION LIST

### Patent Literature

Patent Literature 1: JP 2014-104365A

### DISCLOSURE OF INVENTION

### Technical Problem

A surgical loupe that can adjust a convergence angle more easily has been desired in order to make stereoscopic observation comfortable in surgery performed using a surgical loupe.

### Solution to Problem

According to the present disclosure, there is provided a surgical loupe including: two optical systems that cause images of light from a surgical field which is an observation target to be formed on eyes of a wearer; and a drive unit for adjusting a convergence angle formed by optical axes of the two optical systems.

### Advantageous Effects of Invention

According to the present disclosure described above, a surgical loupe that can adjust a convergence angle more easily can be provided.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is a schematic diagram for describing an example of a configuration outline of a surgical loupe according to an embodiment of the present disclosure.

FIG. 2 is a schematic diagram illustrating an example in which respective optical systems are rotated with respect to the rotational axis of each of the optical systems in a case in which an observation distance to an observation target T is changed.

FIG. 3 is a schematic diagram illustrating an example in which a convergence angle is adjusted by moving optical axis systems.

FIG. 4 is a block diagram illustrating an example of a functional configuration of a surgical loupe 1 according to the embodiment.

FIG. 5 is a block diagram illustrating an example of a configuration of a calibration system 99 according to the embodiment.

FIG. 6 is an explanatory diagram illustrating an example of a position and an attitude of the surgical loupe 1 at the time of calibration according to the embodiment.

FIG. 7 is an explanatory diagram illustrating an example of a position and an attitude of the surgical loupe 1 at the time of calibration according to the embodiment.

FIG. 8 is a flowchart showing an example of calibration of the surgical loupe 1 according to the embodiment.

FIG. 9 is a flowchart showing an example of an operation of the surgical loupe 1 according to the embodiment at the time of observation.

FIG. 10 is an explanatory diagram illustrating an example of a hardware configuration.

### MODE(S) FOR CARRYING OUT THE INVENTION

Hereinafter, (a) preferred embodiment (s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Note that, in this description and the drawings, structural elements that have substantially the same function and structure are sometimes distinguished from each other using different alphabets after the same reference sign. However, when there is no need in particular to distinguish structural elements that have substantially the same function and structure, the same reference sign alone is attached.

Note that description will be provided in the following order.

### <<1. Background>>

<<2. Examples of configuration>>
<2-1. Example of schematic configuration of surgical loupe>
<2-2. Example of functional configuration of surgical loupe>
<2-3. Example of configuration of calibration system>
<<3. Examples of operation>>
<3-1. Example of calibration>
<3-2. Example of operation at time of observation>
<<4. Modified examples>>
<4-1. Modified example 1>
<4-2. Modified example 2>
<<5. Example of hardware configuration>>

### <<6. Conclusion>>

### 1. BACKGROUND

First, before an embodiment of the present disclosure is described, the background to the creation of the present embodiment will be described. Delicate treatments have been demanded in surgical operations, for example, cardiovascular surgery, in recent years. When such surgery is performed, operators need to observe objects in stereoscopic and enlarged views, and thus they often use surgical loupes which are binocular magnifying glasses.

Such a surgical loupe is integrated with, for example, eyeglasses, eyeglass frames, and the like and is customized for each user to be suitable for the visual acuity, the pupillary distance, a desired focal distance of the user, and the like.

In addition, since many surgical loupes have fixed focuses, users have conducted surgery at positions and postures at which the distances between surgical loupes and observation targets (e.g., surgical sites) (which will also be referred to as “observation distances”) are kept constant in order to obtain clear views. Thus, there is concern of increasing burdens on the users, which may be a cause of cervical spondylosis, and the like, for example.

Since surgical loupes have, for example, a focus adjustment function (focus adjustment), the function makes it possible to perform observation even at different observation distances; however, in order to make stereoscopic observation comfortable in such a case, it is desirable to adjust a convergence angle formed by optical axes of two optical systems of a surgical loupe in accordance with an observation distance.

Thus, the present embodiment has been created taking the above circumstance into account as one point. A surgical loupe according to the present embodiment has a drive unit for adjusting a convergence angle formed by optical axes of two optical systems in addition to the focus adjustment function, and thus enables comfortable stereoscopic observation even at different observation distances. In addition, the surgical loupe according to the present embodiment enables comfortable observation even in a case in which an observation distance (a distance to an observation target) is changed during surgery by measuring (sensing) the observation distance and automatically performing focus adjustment and convergence angle adjustment on the basis of the observation distance. Furthermore, the surgical loupe according to the present embodiment enables adjustment to be more suitable for individual users by performing calibration for each user.

Configurations of the surgical loupe according to the present embodiment and a calibration system of the surgical loupe for realizing the above-described effects will be described in detail in that order.

### 2. EXAMPLES OF CONFIGURATION

### 2-1. Example of Schematic Configuration of Surgical Loupe

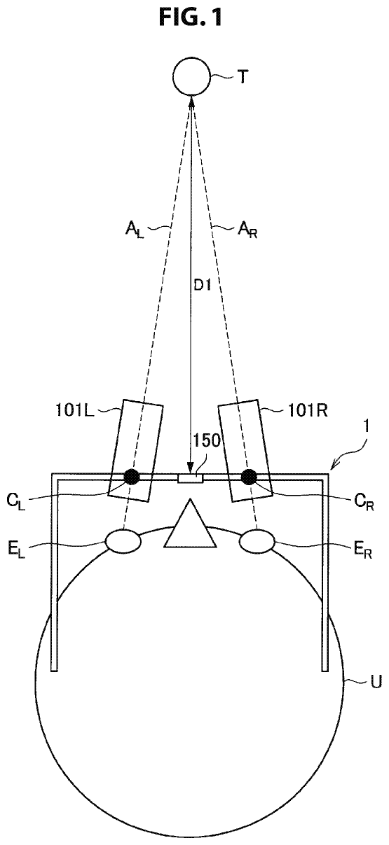

First, an example of a schematic configuration of a surgical loupe according to the present embodiment will be described with reference to FIG. 1 to FIG. 3. FIG. 1 is a schematic diagram for describing an example of a configuration outline of a surgical loupe according to the present embodiment. The user U (wearer) illustrated in FIG. 1 is wearing the surgical loupe 1 according to the present embodiment. The surgical loupe 1 has two optical systems (a left-eye optical system 101L and a right-eye optical system 101R) that cause an image of light from a surgical field, which is an observation target, to be formed on the eyes of the wearer, and a distance measuring sensor unit 150 as illustrated in FIG. 1. Note that the left-eye optical system 101L and the right-eye optical system 101R may be collectively referred to as an optical system 101.

The left-eye optical system 101L causes an image of light from the observation target T to be formed on the left eye E_{L} of the user U and the right-eye optical system 101R causes an image of light from the observation target T to be formed on the right eye E_{R} of the user U.

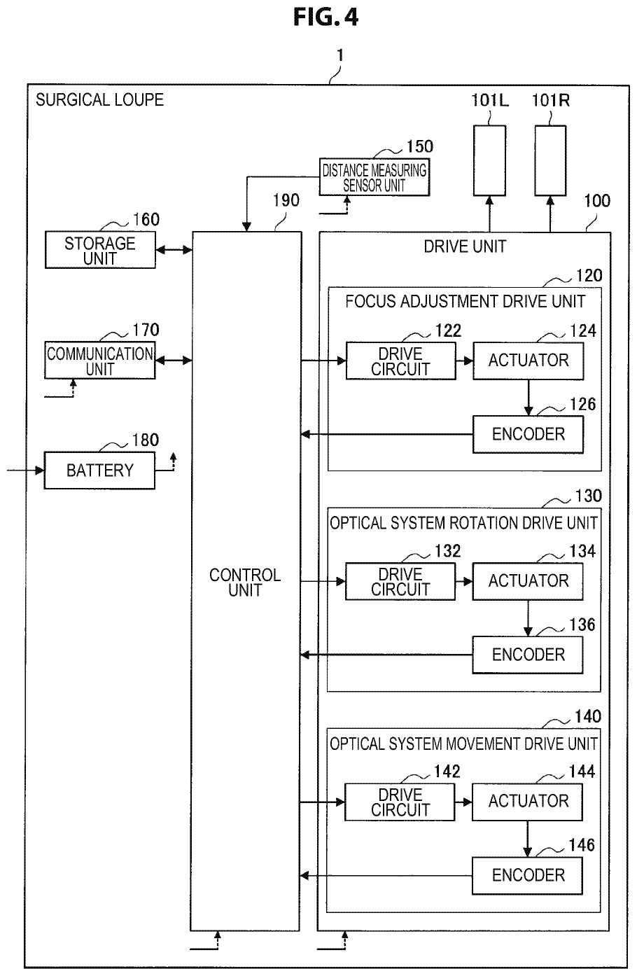

At this time, it is desirable to perform focus adjustment in accordance with the distance from the optical system 101 to the observation target T in order to enable the user U to perform clear observation. Thus, the surgical loupe 1 according to the present embodiment has the distance measuring sensor unit 150 that measures the observation distance to the observation target T (the distance D1 in the example of FIG. 1). In addition, the surgical loupe 1 according to the present embodiment has an autofocus (AF) function of performing automatic focus adjustment by moving a focusing lens (an example of a focusing optical member) included in each of the left-eye optical system 101L and the right-eye optical system 101R in accordance with the observation distance. A functional configuration for realizing the corresponding AF function will be described below with reference to FIG. 4.

In addition, in order to enable the user U to perform comfortable stereoscopic observation, it is desirable to appropriately adjust a convergence angle formed by an optical axis A_{L} of the left-eye optical system 101L and an optical axis A_{R} of the right-eye optical system 101R. For example, the user U can stereoscopically observe the observation target T at the convergence angle at which the optical axis A_{L} and the optical axis A_{R} intersect at the position of the observation target T. Thus, adjusting the convergence angle by, for example, rotating the left-eye optical system 101L and the right-eye optical system 101R with respect to each of a rotational axis C_{L} of the left-eye optical system 101L and a rotational axis C_{R} of the right-eye optical system 101R in accordance with the observation distance is considered.

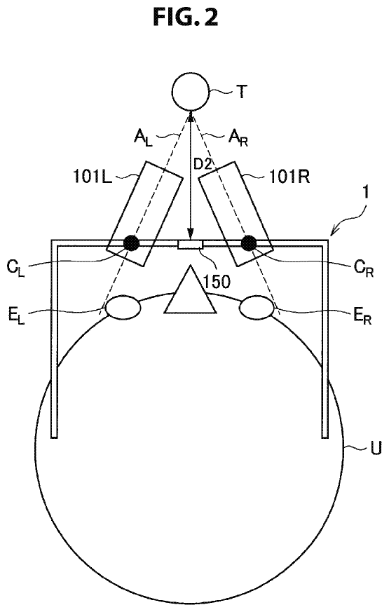

FIG. 2 is a schematic diagram showing an example in which the respective optical systems are rotated with respect to the rotational axis of each of the optical systems in a case in which an observation distance to the observation target T is changed. In the example illustrated in FIG. 2, an observation distance D2 to the observation target T is shorter than the observation distance D1 of the example illustrated in FIG. 1, and the convergence angle formed by the optical axis A_{L} and the optical axis A_{R} by rotating the respective optical systems with respect to the rotational axis of each of the optical systems is larger than that of the example illustrated in FIG. 1. At this time, if the observation distance is short, an image of light from the observation target T is not formed on the left eye E_{L} and the right eye E_{R} of the user U and there may be a case in which the user U is not able to observe the observation target T. In the example illustrated in FIG. 2, for example, the optical axis A_{L} of the left-eye optical system 101L and the optical axis A_{R} of the right-eye optical system 101R do not intersect the left eye E_{L} and the right eye E_{R} of the user U.

Thus, the surgical loupe 1 according to the present embodiment adjusts the convergence angle by moving the optical axis systems, in addition to adjusting the convergence angle through rotation as described above. The convergence angle can be adjusted by, for example, moving each of the optical axis systems and thus changing the distance between the rotational axis C_{L} of the left-eye optical system 101L and the rotational axis C_{R} of the right-eye optical system 101R.

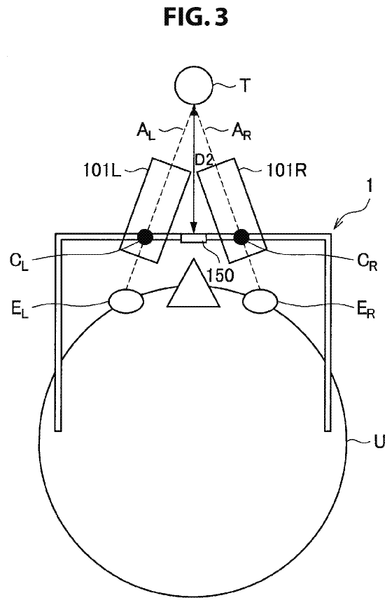

FIG. 3 is a schematic diagram illustrating an example in which a convergence angle is adjusted by moving the optical axis systems. In the example illustrated in FIG. 3, the observation distance to the observation target T is the same as the observation distance D2 illustrated in FIG. 2; however, light from the observation target T is transmitted through each of the optical systems and an image thereof can be formed on the left eye E_{L} and the right eye E_{R} of the user U when the two optical systems are moved and thus the distance between the rotational axes of the two optical systems is shortened.

The example of the schematic configuration of the surgical loupe 1 according to the present embodiment has been described above. Note that the surgical loupe 1 illustrated in FIG. 1 to FIG. 3 is an example and the present embodiment is not limited thereto. For example, the distance measuring sensor unit 150 may be disposed at a different position from that in the examples illustrated in FIG. 1 to FIG. 3.

### 2-2. Example of Functional Configuration of Surgical Loupe

Next, an example of a functional configuration of the surgical loupe 1 according to the present embodiment will be described with reference to FIG. 4. Note that the rotational axes (the rotational axis C_{L} and the rotational axis C_{R}) of the optical systems described with reference to FIG. 1 to FIG. 3 will be appropriately referred to in the following description.

FIG. 4 is a block diagram illustrating an example of a functional configuration of the surgical loupe 1. The surgical loupe according to the present embodiment has a drive unit 100, the left-eye optical system 101L, the right-eye optical system 101R, the distance measuring sensor unit 150, a storage unit 160, the communication unit 170, a battery 180, and a control unit 190 as illustrated in FIG. 4.

The drive unit 100 is controlled by the control unit 190, which will be described below, to operate focus adjustment of the two optical systems (the left-eye optical system 101L and the right-eye optical system 101R) described with reference to FIG. 1 to FIG. 3 and adjustment of the convergence angle formed by the optical axes of the two optical systems. For example, the drive unit 100 functions as a focus adjustment drive unit 120, an optical system rotation drive unit 130, and an optical system movement drive unit 140 as illustrated in FIG. 4.

The focus adjustment drive unit 120 causes a focusing lens (an example of an optical member for focusing) of each optical system to move for focus adjustment. The focus adjustment drive unit 120 can include, for example, a drive circuit 122, an actuator 124, and an encoder 126 as illustrated in FIG. 4. The drive circuit 122 causes the actuator 124 to be driven by supplying a current to the actuator 124 in accordance with control of the control unit 190. The actuator 124 causes the focusing lens included in each of the left-eye optical system 101L and the right-eye optical system 101R to move in accordance with the applied current. The encoder 126 is a sensor that detects a position (a movement amount) of the focusing lens moved by the actuator 124. The encoder 126 supplies the detected position of the focusing lens to the control unit 190.

With the above configuration, focus adjustment can be performed and thus the user can perform clear observation even at different observation distances.

The optical system rotation drive unit 130 causes the optical systems to rotate with respect to the rotational axis of each of the optical systems to adjust the convergence angle. The optical system rotation drive unit 130 can include, for example, a drive circuit 132, an actuator 134, and an encoder 136 as illustrated in FIG. 4. The drive circuit 132 causes the actuator 134 to be driven by supplying a current to the actuator 134 in accordance with control of the control unit 190. The actuator 134 causes the left-eye optical system 101L and the right-eye optical system 101R illustrated in FIG. 1 to rotate with respect to the rotational axis C_{L} and the rotational axis C_{R} respectively in accordance with the applied current. The encoder 136 is a sensor that detects rotation angles of the rotational axis C_{L} and the rotational axis C_{R} rotated by the actuator 134. The encoder 136 provides the detected rotation angles to the control unit 190.

The optical system movement drive unit 140 causes each of the optical systems to move to adjust the convergence angle. For example, the optical system movement drive unit 140 may cause the left-eye optical system 101L and the right-eye optical system 101R to move to change the distance between the rotational axis C_{L} of the left-eye optical system 101L and the rotational axis C_{R} of the right-eye optical system 101R as described with reference to FIG. 1 to FIG. 3. The optical system movement drive unit 140 can include, for example, a drive circuit 142, an actuator 144, and an encoder 146 as illustrated in FIG. 4. The drive circuit 142 causes the actuator 144 to be driven by supplying a current to the actuator 144 in accordance with control of the control unit 190. The actuator 144 causes the left-eye optical system 101L and the right-eye optical system 101R illustrated in FIG. 1 to rotate with respect to the rotational axis C_{L} and the rotational axis C_{R} respectively in accordance with the applied current. The encoder 146 is a sensor that detects rotation angles of the rotational axis C_{L} and the rotational axis C_{R} rotated by the actuator 144. The encoder 146 provides the detected rotation angles to the control unit 190.

With the above configuration, the convergence angle can be adjusted, and the user can stereoscopically observe the observation target. In addition, since the adjustment of the convergence angle through the movement of the optical systems is possible, in addition to the rotation of the optical systems, the convergence angle can be adjusted so that an image of light from the observation target is formed on the eyes of the user.

The distance measuring sensor unit 150 is a sensor that measures (senses) the distance to the observation target (observation distance). The distance measuring sensor unit 150 supplies the observation distance acquired from the measurement to the control unit 190.

The storage unit 160 stores a program and data for causing each configuration of the surgical loupe 1 to function. For example, the storage unit 160 stores parameters to be used by the control unit 190, which will be described below, to control the drive unit 100. Note that the parameters may be obtained from calibration performed by each user using a calibration system, which will be described below, or may be parameters serving as references (e.g., initial values) for control of the drive unit 100. In addition, the storage unit 160 may store an observation distance at the time of calibration (which may be referred to as a reference observation distance).

The communication unit 170 is a communication interface that mediates communication with other devices. The communication unit 170 supports an arbitrary wireless communication protocol or wired communication protocol and establishes a communication connection with other devices. In addition, the communication unit 170 performs transmission or reception of information in accordance with control of the control unit 190.

The battery 180 supplies power to each block of the surgical loupe 1 illustrated in FIG. 4 via power supply lines that are partially illustrated with dashed lines in the drawing.

The control unit 190 controls each configuration of the surgical loupe 1. For example, the control unit 190 controls the communication unit 170 such that communication with (transmission to or reception from) other devices is controlled. In addition, the control unit 190 may store a parameter received via the communication unit 170 in the storage unit 160. In addition, the control unit 190 controls the drive unit 100. The control unit 190 may control the drive unit 100, for example, on the basis of an observation distance measured by the distance measuring sensor unit 150, or may control the drive unit 100 on the basis of information received from another device via the communication unit 170.

The control unit 190 can realize the autofocus (AF) function of automatically performing focus adjustment by controlling the focus adjustment drive unit 120 such that the observation target is focused, for example, in accordance with an observation distance.

Here, for example, since the distance from the principal point of the optical system to the eyes of the user (focal distance) can vary depending on users, a proper position of the focusing lens can be different among the users even at the same observation distance. Thus, the control unit 190 may control the focus adjustment drive unit 120 further on the basis of a parameter obtained from calibration, which will be described below, and stored in the storage unit 160. Note that, in such a case, the control unit 190 may perform control further based on the observation distance at the time of calibration (a reference observation distance) or control the focus adjustment drive unit 120 on the basis of, for example, the difference between the reference observation distance and the current observation distance. With this configuration, focus adjustment can be performed with higher accuracy depending on users.

In addition, the control unit 190 may control the optical system rotation drive unit 130 and the optical system movement drive unit 140 such that the optical axes of each of the optical systems intersect the observation target in accordance with the observation distance. For example, the control unit 190 may control the optical system rotation drive unit 130 such that the left-eye optical system 101L and the right-eye optical system 101R rotate more inward as the observation distance becomes shorter. In addition, the control unit 190 may control the optical system movement drive unit 140 such that the distance between the rotational axis C_{L} of the left-eye optical system 101L and the rotational axis C_{R} of the right-eye optical system 101R is decreased more as the observation distance becomes shorter.

With the above configuration, even in a case in which the observation distance is changed during surgery, the convergence angle is automatically adjusted, and thus the user can perform comfortable stereoscopic observation.

Note that, directions of the optical axes of each of the optical systems can be changed depending on both rotation and movement of each of the optical systems. Thus, a control method for causing the optical axes of each of the optical systems to intersect the observation target is not limited to the above-described example, and the control unit 190 can control the optical system rotation drive unit 130 and the optical system movement drive unit 140 in various ways.

In addition, since the distance between both eyes can be different among users, for example, proper positions and rotation angles of the optical systems can be different among the users even at the same observation distance. Thus, the control unit 190 may control the optical system rotation drive unit 130 and the optical system movement drive unit 140 further on the basis of a parameter obtained from calibration, which will be described below, and stored in the storage unit 160. Note that, in such a case, the control unit 190 may perform the control further on the basis of the observation distance at the time of the calibration (the reference observation distance), or may control the optical system rotation drive unit 130 and the optical system movement drive unit 140 in accordance with, for example, the difference between the reference observation distance and the current observation distance. With this configuration, it is possible to adjust the convergence angle with high accuracy in accordance with the user.

### 2-3. Example of Configuration of Calibration System

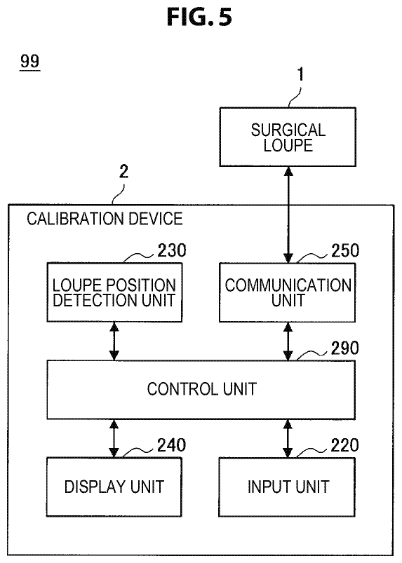

The example of the functional configuration of the surgical loupe 1 according to the present embodiment has been described above. Next, an example of a configuration of a calibration system for performing calibration according to the above-described surgical loupe 1 will be described with reference to FIG. 5.

FIG. 5 is a block diagram illustrating an example of a configuration of a calibration system 99 according to the present embodiment. The calibration system 99 according to the present embodiment includes the surgical loupe 1 and a calibration device 2 as illustrated in FIG. 5. Since the surgical loupe 1 illustrated in FIG. 5 has been described with reference to FIG. 1 to FIG. 4, description thereof will be omitted here.

The calibration device 2 is an information processing apparatus having an input unit 220, a loupe position detection unit 230, a display unit 240, a communication unit 250, and a control unit 290 as illustrated in FIG. 5.

The input unit 220 is an interface that receives an input of a user. For example, the user wearing the surgical loupe 1 can input information indicating whether comfortable observation is possible through the input unit 220. In addition, the user wearing the surgical loupe may perform operations for focus adjustment and convergence angle adjustment of the surgical loupe 1 that is performing calibration through the input unit 220.

The loupe position detection unit 230 detects a position and an attitude of the surgical loupe 1. For example, the loupe position detection unit 230 may include a camera and detect a position and an attitude of the surgical loupe 1 on the basis of an image containing the surgical loupe 1 acquired by the camera. In addition, in a case in which a marker reflecting infrared light is mounted on the surgical loupe 1, the loupe position detection unit 230 may include an infrared light output device and an infrared light sensor and detect a position and an attitude of the surgical loupe 1 through detection of the marker.

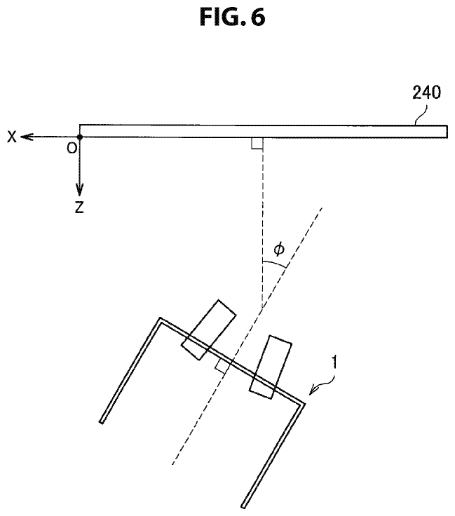

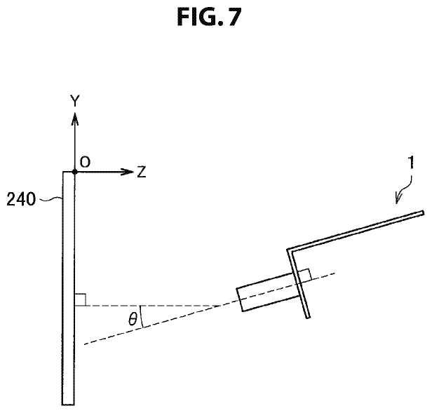

Note that a position and an attitude of the surgical loupe 1 may be information expressed with respect to the display unit 240. FIG. 6 and FIG. 7 are explanatory diagrams illustrating an example of a position and an attitude of the surgical loupe 1 at the time of calibration. Note that FIG. 6 is a plan view, and FIG. 7 is a side view. The position and the attitude of the surgical loupe 1 can be expressed with, for example, a position in the X direction, a position in the Y direction, a position in the Z direction, an angle φ, and angle θ illustrated in FIG. 6 and FIG. 7 with respect to the display unit 240.

Note that detection of a position and an attitude of the surgical loupe 1 is not limited to the above-described example, and a position and an attitude of the surgical loupe 1 can be detected using various means.

The display unit 240 is a display that performs display in accordance with control by the control unit 290. The display unit 240 may display an image for calibration or display, for example, a predetermined geometric pattern.

In addition, the display unit 240 may display guidance on calibration or the like to the user. With this configuration, the user can perform calibration at a proper position or perform calibration with efficiency.

The communication unit 250 is a communication interface that mediates communication with other devices. The communication unit 250 supports an arbitrary wireless communication protocol or wired communication protocol and establishes, for example, a communication connection with the surgical loupe 1. In addition, the communication unit 250 can transmit control information relating to focus adjustment and convergence angle adjustment of the surgical loupe 1 that is performing calibration, information indicating that calibration has been completed, or the like to the surgical loupe 1 in accordance with control of the control unit 290.

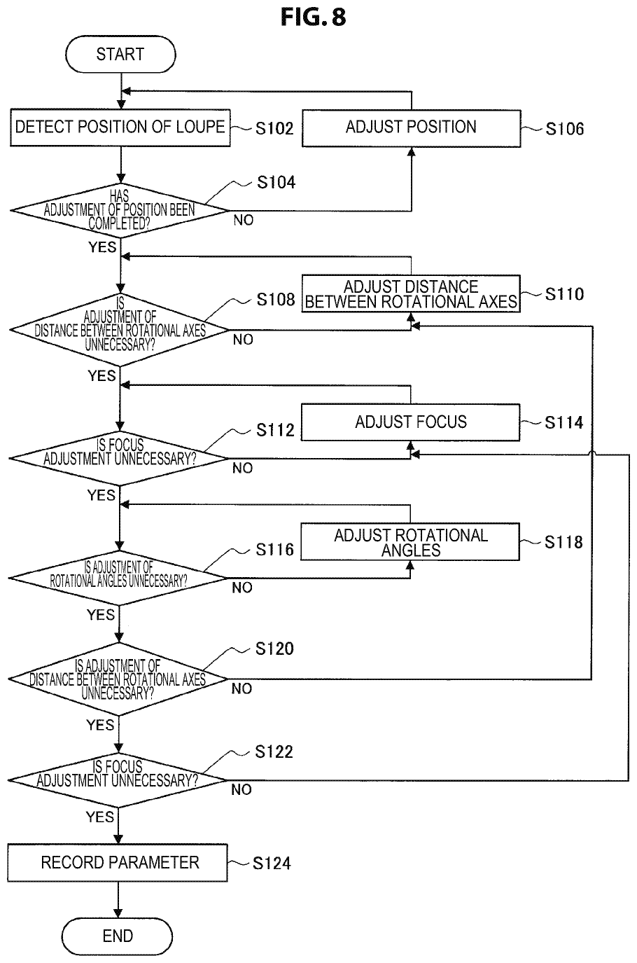

The control unit 290 controls each of the configurations of the calibration device 2 illustrated in FIG. 5. With control by the control unit 290, for example, calibration of the surgical loupe 1 can be executed for each of users. Note that an example of calibration that can be executed under control of the control unit 290 will be described below with reference to FIG. 8.

### 3. EXAMPLES OF OPERATION

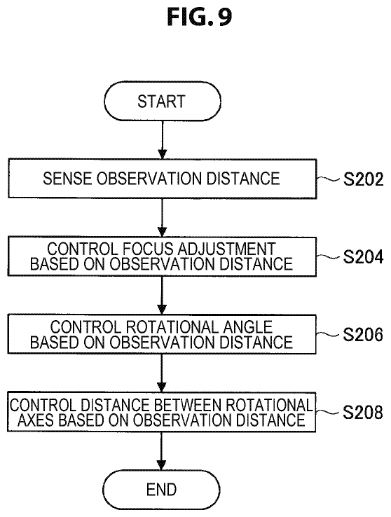

The examples of the configurations according to the present embodiment have been described above. Next, examples of operations according to the present embodiment will be described. After an example of calibration using the calibration system 99 is described below with reference to FIG. 8, and an example of an operation of the surgical loupe 1 at the time of observation will be described with reference to FIG. 9.

### 3-1. Example of Calibration

FIG. 8 is a flowchart showing an example of calibration of the surgical loupe 1 according to the present embodiment. First, the loupe position detection unit 230 detects a position and an attitude of the surgical loupe 1 with respect to the display unit 240 (S102) as illustrated in FIG. 8.

Next, the position of the surgical loupe 1 is adjusted until the position adjustment is completed (S108). For example, the position adjustment may be performed to get a predetermined observation distance and angle, and the determination of Step S104 may be automatically made by the control unit 290 or on the basis of a user input.

In a case in which the position adjustment has been completed (YES in S104), the necessity/non-necessity of adjustment of the distance between the rotational axes of the optical systems is determined (S108). The determination of Step S108 may be made, for example, on the basis of a user input. In a case in which the adjustment of the distance between the rotational axes is determined to be necessary (NO in S108), the optical system movement drive unit 140 is controlled such that the optical systems are moved and thus the distance between the rotational axes is adjusted (S110). Note that the control of the optical system movement drive unit 140 may be performed, for example, on the basis of a user input, or may be automatically performed by the control unit 290 or the control unit 190. The adjustment of the distance between the rotational axes can be performed until the adjustment of the distance between the rotational axes is determined to be unnecessary in Step S108.

In a case in which the adjustment of the distance between the rotational axes is determined to be unnecessary (YES in S108), the necessity/non-necessity of focus adjustment is determined (S112). The determination of Step S112 may be made, for example, on the basis of a user input. In a case in which focus adjustment is determined to be necessary (NO in S112), the focus adjustment drive unit 120 is controlled such that the focusing lenses included in the optical systems are moved and thus focus adjustment is performed (S114). Note that the control of the focus adjustment drive unit 120 may be performed, for example, on the basis of a user input or may be automatically performed by the control unit 290 or the control unit 190. The focus adjustment can be performed until the focus adjustment is determined to be unnecessary in Step S112.

In a case in which the focus adjustment is determined to be unnecessary (YES in S112), necessity/non-necessity of adjustment of rotational angles of the optical systems is determined (S116). The determination of Step S116 may be made, for example, on the basis of a user input. In a case in which the adjustment of the rotational angles of the optical systems is determined to be necessary (NO in S116), the optical system rotation drive unit 130 is controlled such that the angles of each of the optical systems with respect to the rotational axes is adjusted (S118). Note that the control of the optical system rotation drive unit 130 may be performed, for example, on the basis of a user input or may be automatically performed by the control unit 290 or the control unit 190. The adjustment of the rotational angles of the optical systems can be performed until the adjustment of the rotational angles of the optical systems is determined to be unnecessary in Step S116.

Since control of the three focus adjustment drive unit 120, the optical system rotation drive unit 130, and the optical system movement drive unit 140 is involved with whether comfortable observation is possible for the user, it is necessary to recursively perform the operation.

In a case in which the adjustment of the rotational angles is determined to be unnecessary (YES in S116), necessity/non-necessity of adjustment of the distance between the rotational axes of the optical systems is determined once again (S120). The determination of Step S120 may be made, for example, on the basis of a user input. Here, in a case in which it is determined that adjustment of the distance between the rotational axes of the optical systems is necessary (NO in S120), the process returns to Step S110.

In a case in which it is determined that the adjustment of the distance between the rotational axes of the optical systems is unnecessary (YES in S120), necessary/non-necessity of focus adjustment is determined once again (S122). The determination of Step S122 may be made, for example, on the basis of a user input. Here, in a case in which it is determined that focus adjustment is necessary (NO in S122), the process returns to Step S114.

In a case in which it is determined that focus adjustment is unnecessary (YES in S122), the control unit 290 controls the communication unit 250 such that, for example, information for notifying that calibration has been completed is transmitted to the surgical loupe 1. Then, the control unit 190 of the surgical loupe 1 causes parameters relating to control of the focus adjustment drive unit 120, the optical system rotation drive unit 130, and the optical system movement drive unit 140 of the current time to be stored in the storage unit 160.

Note that the method for calibration illustrated in FIG. 8 is an example and is not limited to the example according to the present embodiment, and calibration can be performed in various methods in which proper parameters for each user can be acquired.

### 3-2. Example of Operation at Time of Observation

Next, an example of an operation of the surgical loupe 1 at the time of observation will be described with reference to FIG. 9. FIG. 9 is a flowchart showing an example of an operation of the surgical loupe 1 at the time of observation. Note that the processes shown in FIG. 9 can be performed later than the calibration described with reference to FIG. 8.

First, the distance measuring sensor unit 150 senses (measures) the observation distance that is the distance to the observation target (S202) as illustrated in FIG. 9. Next, the control unit 190 controls the focus adjustment drive unit 120 on the basis of the observation distance (S204). Next, the control unit 190 controls the optical system rotation drive unit 130 on the basis of the observation distance (S206). Next, the control unit 190 controls the optical system movement drive unit 140 on the basis of the observation distance (S208).

Note that the series of operations illustrated in FIG. 9 may be appropriately repeated. In addition, the flowchart diagram illustrated in FIG. 9 is merely an example and is not limited to the example. For example, control of Steps S204 to S208 may be performed in a different order or may be performed in parallel.

### 4. MODIFIED EXAMPLES

An embodiment of the present disclosure has been described above. Several modified examples of an embodiment of the present disclosure will be described below. Note that each of the modified examples to be described below may be applied to an embodiment of the present disclosure alone or in combination. In addition, each of the modified examples may be applied instead of or in addition to the configuration described in the embodiment of the present disclosure.

### 4-1. Modified Example 1

In the above-described embodiment, the example in which a parameter obtained from calibration for each user is stored in the storage unit 160 has been described. Here, the storage unit 160 may store not only a parameter for one user but also parameters for a plurality of users. For example, the storage unit 160 may store the users and the parameters in association.

In addition, in that case, the surgical loupe 1 may have a user identification function and the control unit 190 may control the drive unit 100 on the basis of a parameter corresponding to an identified user.

The control unit 190 may, for example, acquire information of a user from another device, which is not illustrated, via the communication unit 170 and identify a user on the basis of the information of the user.

For example, information of a current or future operator in charge (an example of the information of a user) may be acquired from an external server managing information relating to the surgery. In addition, in a case in which each user possesses a communicable ID card or a communication device (a mobile telephone, a smartphone, etc.) containing information of a user, the control unit 190 may acquire the information of the user from the ID card or the communication device via the communication unit 170.

In addition, the surgical loupe 1 may further have an input function and thus a user may be identified on the basis of a user input. In addition, the surgical loupe 1 may further have a sensor that performs sensing of information of a user such as a fingerprint, or an iris of the user and a user may be identified on the basis of sensing of the sensor.

According to the configuration, in a case in which a plurality of users use the surgical loupe 1, a user who has performed calibration before can use the surgical loupe 1 without performing calibration again. Note that a user identification method is not limited to the above-described example, and identification can be performed in various methods.

### 4-2. Modified Example 2

The surgical loupe 1 may have a headlight (lighting unit). In addition, the control unit 190 may control an amount of light, a range of irradiation, or the like according to the lighting unit in accordance with an observation distance.

The control unit 190 may perform, for example control of the lighting unit such that the amount of light increases as the observation distance becomes longer. In addition, the control unit 190 may perform control of the lighting unit such that the angle of irradiation becomes narrower as the observation distance becomes longer.

With the above configuration, an observation target can be satisfactorily irradiated and the user can comfortably observe the observation target. In addition, power consumption of the lighting unit can be satisfactorily controlled due to the control of the lighting unit based on the observation distance.

### 5. EXAMPLE OF HARDWARE CONFIGURATION

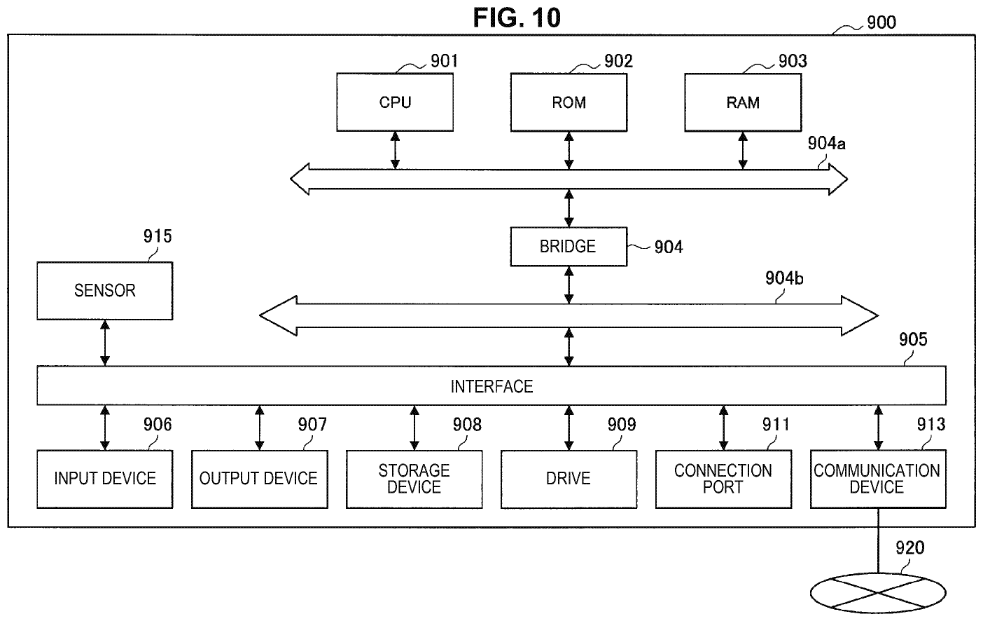

The embodiment of the present disclosure has been described hitherto. Finally, a hardware configuration of an information processing apparatus according to the present embodiment of the present disclosure will be described with reference to FIG. 10. FIG. 10 is a block diagram illustrating an example of the hardware configuration of the information processing apparatus according to the present embodiment of the present disclosure. Note that the information processing apparatus 900 illustrated in FIG. 10 can realize, for example, the surgical loupe 1 or the calibration device 2 each illustrated in FIG. 4 or FIG. 5. Information processing by the surgical loupe 1 and the calibration device 2 according to the present embodiment is realized in cooperation with software and hardware which will be described below.

As illustrated in FIG. 10, the information processing apparatus 900 includes a central processing unit (CPU) 901, a read only memory (ROM) 902, a random access memory (RAM) 903, and a host bus 904a. In addition, the information processing apparatus 900 includes a bridge 904, an external bus 904b, an interface 905, an input device 906, an output device 907, a storage device 908, a drive 909, a connection port 911, a communication device 913, and a sensor 915. The information processing apparatus 900 may include a processing circuit such as a DSP or an ASIC instead of the CPU 901 or along therewith.

The CPU 901 functions as an arithmetic processing device and a control device and controls the overall operation in the information processing apparatus 900 according to various programs. Further, the CPU 901 may be a microprocessor. The ROM 902 stores programs, operation parameters, and the like used by the CPU 901. The RAM 903 temporarily stores programs used in execution of the CPU 901, parameters appropriately changed in the execution, and the like. The CPU 901 may form the control unit 190, and the control unit 290, for example.

The CPU 901, the ROM 902, and the RAM 903 are connected by the host bus 904a including a CPU bus and the like. The host bus 904a is connected with the external bus 904b such as a peripheral component interconnect/interface (PCI) bus via the bridge 904. Further, the host bus 904a, the bridge 904, and the external bus 904b are not necessarily separately configured and such functions may be mounted in a single bus.

The input device 906 is realized by a device through which a user inputs information, such as a mouse, a keyboard, a touch panel, a button, a microphone, a switch, and a lever, for example. In addition, the input device 906 may be a remote control device using infrared ray or other electric waves, or external connection equipment such as a cellular phone or a PDA corresponding to an operation of the information processing apparatus 900, for example. Furthermore, the input device 906 may include an input control circuit or the like which generates an input signal on the basis of information input by the user using the aforementioned input means and outputs the input signal to the CPU 901, for example. The user of the information processing apparatus 900 may input various types of data or order a processing operation for the information processing apparatus 900 by operating the input device 906. The input device 906 may form the input unit 220, for example.

The output device 907 is formed by a device that may visually or aurally notify the user of acquired information. As such devices, there are a display device such as a CRT display device, a liquid crystal display device, a plasma display device, an EL display device, or a lamp, a sound output device such as a speaker and a headphone, a printer device, and the like. The output device 907 outputs results acquired through various processes performed by the information processing apparatus 900, for example. Specifically, the display device visually displays results acquired through various processes performed by the information processing apparatus 900 in various forms such as text, images, tables, and graphs. On the other hand, the sound output device converts audio signals including reproduced sound data, audio data, and the like into analog signals and aurally outputs the analog signals. The output device 907 may form the display unit 240, for example.

The storage device 908 is a device for data storage, formed as an example of a storage unit of the information processing apparatus 900. For example, the storage device 908 is realized by a magnetic storage device such as an HDD, a semiconductor storage device, an optical storage device, a magneto-optical storage device, or the like. The storage device 908 may include a storage medium, a recording device for recording data on the storage medium, a reading device for reading data from the storage medium, a deletion device for deleting data recorded on the storage medium, and the like. The storage device 908 stores programs and various types of data executed by the CPU 901, various types of data acquired from the outside, and the like. The storage device 908 may form the storage unit 160, for example.

The drive 909 is a reader/writer for storage media and is included in or externally attached to the information processing apparatus 900. The drive 909 reads information recorded on a removable storage medium such as a magnetic disc, an optical disc, a magneto-optical disc, or a semiconductor memory mounted thereon, and outputs the information to the RAM 903. In addition, the drive 909 may write information regarding the removable storage medium.

The connection port 911 is an interface connected with external equipment and is a connector to the external equipment through which data may be transmitted through a universal serial bus (USB) and the like, for example.

The communication device 913 is a communication interface formed by a communication device for connection to a network 920 or the like, for example. The communication device 913 is a communication card or the like for a wired or wireless local area network (LAN), long term evolution (LTE), Bluetooth (registered trademark), or wireless USB (WUSB), for example. In addition, the communication device 913 may be a router for optical communication, a router for asymmetric digital subscriber line (ADSL), various communication modems, or the like. For example, the communication device 913 may transmit/receive signals and the like to/from the Internet and other communication apparatuses according to a predetermined protocol such as, for example, TCP/IP. The communication device 913 may form the communication unit 170, and the communication unit 250, for example.

The sensor 915 corresponds to various types of sensors such as an acceleration sensor, a gyro sensor, a geomagnetic sensor, a light sensor, a sound sensor, a distance measuring sensor, and a force sensor, for example. The sensor 915 acquires information regarding a state of the information processing apparatus 900 itself, such as an attitude and a movement speed of the information processing apparatus 900, and information regarding a surrounding environment of the information processing apparatus 900, such as brightness and noise of the periphery of the information processing apparatus 900. In addition, the sensor 915 may include a GPS sensor that receives a GPS signal, and measures latitude, longitude, and altitude of the device. The sensor 915 may form, for example, the distance measuring sensor unit 150.

Further, the network 920 is a wired or wireless transmission path of information transmitted from devices connected to the network 920. For example, the network 920 may include a public circuit network such as the Internet, a telephone circuit network, or a satellite communication network, various local area networks (LANs) including Ethernet (registered trademark), a wide area network (WAN), and the like. In addition, the network 920 may include a dedicated circuit network such as an internet protocol-virtual private network (IP-VPN).

Hereinbefore, an example of a hardware configuration capable of realizing the functions of the information processing apparatus 900 according to this embodiment is shown. The respective components may be implemented using universal members, or may be implemented by hardware specific to the functions of the respective components. Accordingly, according to a technical level at the time when the embodiments are executed, it is possible to appropriately change hardware configurations to be used.

In addition, a computer program for realizing each of the functions of the information processing apparatus 900 according to the present embodiment as described above may be created, and may be mounted in a PC or the like. Furthermore, a computer-readable recording medium on which such a computer program is stored may be provided. The recording medium is a magnetic disc, an optical disc, a magneto-optical disc, a flash memory, or the like, for example. Further, the computer program may be delivered through a network, for example, without using the recording medium. In addition, the above-described computer program may be distributed through, for example, a network without using a recording medium.

### 6. CONCLUSION

According to the embodiment of the present disclosure, the surgical loupe that can adjust a convergence angle more easily is provided as described above. In addition, the surgical loupe according to the embodiment of the present disclosure has the drive unit for adjusting a convergence angle formed by the optical axes of the two optical systems in addition to the focus adjustment function, and thus comfortable stereoscopic observation is possible even at different observation distances. Furthermore, the surgical loupe according to the present embodiment automatically performs focus adjustment and convergence angle adjustment on the basis of an observation distance, and thus even in a case in which an observation distance is changed during surgery, comfortable observation is possible. Thus, for example, a user can observe an observation target at a close distance in a case in which the user wants an enlarged view and at a remote distance in a case in which the user wants an overhead view, and thus observation with a higher degree of freedom is possible. Furthermore, the surgical loupe according to the present embodiment enables adjustment more suitable for each user by performing calibration for each user.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

A surgical loupe including:

two optical systems that cause images of light from a surgical field which is an observation target to be formed on eyes of a wearer; and

a drive unit for adjusting a convergence angle formed by optical axes of the two optical systems.

(2)

The surgical loupe according to (1), in which the drive unit causes the optical systems to rotate with respect to rotational axes of the optical systems to adjust the convergence angle.

(3)

The surgical loupe according to (1) or (2), in which the drive unit causes the optical systems to move to adjust the convergence angle.

(4)

The surgical loupe according to any one of (1) to (3), in which the drive unit causes an optical member for focusing included in the optical systems to move to further perform focus adjustment.

(5)

The surgical loupe according to any one of (1) to (4), further including:

a control unit that controls the drive unit.

(6)

The surgical loupe according to (5), further including:

a distance measuring sensor unit that measures a distance to an observation target, in which the control unit controls the drive unit on the basis of the distance.

(7)

The surgical loupe according to (6), further including:

a lighting unit, in which the control unit controls the lighting unit on the basis of the distance.

(8)

The surgical loupe according to any one of (5) to (7), further including:

a storage unit that stores a parameter that serves as a reference for control of the drive unit,

in which the control unit controls the drive unit on the basis of the parameter.

(9)

The surgical loupe according to (8),

in which the storage unit stores the parameter in association with a user, and the control unit controls the drive unit on the basis of the parameter corresponding to an identified user.

(10)

The surgical loupe according to (9), in which the control unit identifies the user on the basis of information of the user acquired from another device.

(11)

The surgical loupe according to any one of (8) to (10), in which the parameter is obtained through calibration for each user.

### REFERENCE SIGNS LIST

1 surgical loupe
2 calibration device
99 calibration system
100 drive unit
101L left-eye optical system
101R right eye optical system
120 focus adjustment drive unit
130 optical system rotation drive unit
140 optical system movement drive unit
150 distance measuring sensor unit
160 storage unit
170 communication unit
180 battery
190 control unit
220 input unit
230 loupe position detection unit
240 display unit
250 communication unit
290 control unit

1. A surgical loupe comprising:
two optical systems that cause images of light from a surgical field which is an observation target to be formed on eyes of a wearer; and
a drive unit for adjusting a convergence angle formed by optical axes of the two optical systems.

2. The surgical loupe according to claim 1, wherein the drive unit causes the optical systems to rotate with respect to rotational axes of the optical systems to adjust the convergence angle.

3. The surgical loupe according to claim 1, wherein the drive unit causes the optical systems to move to adjust the convergence angle.

4. The surgical loupe according to claim 1, wherein the drive unit causes an optical member for focusing included in the optical systems to move to further perform focus adjustment.

5. The surgical loupe according to claim 1, further comprising:
a control unit that controls the drive unit.

6. The surgical loupe according to claim 5, further comprising:
a distance measuring sensor unit that measures a distance to an observation target,
wherein the control unit controls the drive unit on a basis of the distance.

7. The surgical loupe according to claim 6, further comprising:
a lighting unit,
wherein the control unit controls the lighting unit on the basis of the distance.

8. The surgical loupe according to claim 5, further comprising:
a storage unit that stores a parameter that serves as a reference for control of the drive unit,
wherein the control unit controls the drive unit on a basis of the parameter.

9. The surgical loupe according to claim 8,
wherein the storage unit stores the parameter in association with a user, and
the control unit controls the drive unit on a basis of the parameter corresponding to an identified user.

10. The surgical loupe according to claim 9, wherein the control unit identifies the user on a basis of information of the user acquired from another device.

11. The surgical loupe according to claim 8, wherein the parameter is obtained through calibration for each user.
